(19) United States
(12) Patent Application Publication
(10) Pub. No.: US 2008/0255593 A1
(43) Pub. Date: Oct. 16, 2008

(54) Prosthetic repair patch with suture retaining structure

(76) Inventor: Pascal St-Germain, Bathurst (CA)

Correspondence Address: Franz BONSANG;c/o PROTECTIONS EQUINOX INC., Suite 410, 1500, Du College, St-Laurent, QC, H4L 5G6, CA

(21) Appl. No.: 11/783,757

(22) Filed: Apr. 12, 2007

(51) Int. Cl. A61F 2/02

(52) U.S. Cl. 606/151

(57) ABSTRACT

A prosthetic repair patch has a sheet and a plurality of retaining structures connected thereto. The sheet, with first and second sheet surfaces, completely under covers a hernia in tissue of a patient with the first sheet surface adjacently abutting a first surface of the tissue that faces away from a person installing the patch. The retaining structures are connected to the sheet in a spaced apart configuration and are configured for retaining a suture passed therethrough, each suture being initially passed from the second tissue surface through the first tissue surface, through at least one retaining structure, and back through the first tissue surface and out of the second tissue surface. Suture ends of each suture may then be drawn away from the second tissue surface to draw the first sheet surface into local and adjacent abutment with the first tissue surface and attached to one another.

### FIELD OF THE INVENTION

The present invention relates to prosthetic repair patches for repairing undesired apertures, such as hernias, in biological tissue of the abdominal wall of a patient, and is more particularly concerned with a prosthetic repair patch having a suture retaining structure.

### BACKGROUND OF THE INVENTION

It is well known in the art to use prosthetic repair patches to repair, by under covering, undesired apertures, such as hernias, in biological tissue of the abdominal wall, aponeurosis or the like of a patient with prosthetic repair patches. Typically, such patches are made of biologically compatible material and are surgically placed under the hernia and then connected to the abdominal wall surrounding the hernia using sutures.

An example of such a prosthetic repair patch is described in U.S. Pat. No. 6,120,539, issued to Eldridge et al. The patch described therein comprises a sheet used for, among other things, repair of ventral hernias, in patients by placement of the patch under the hernia with a first sheet surface thereof in adjacent abutment to the surrounding tissue, typically a first tissue surface which faces away from the health professional that is placing the patch in the patient to repair the hernia. The advantages of using such patches, as opposed to other approaches for repairing hernias, are generally well known in medical arts, and include, notably, reduced risk of hernia reoccurrence. Such patches are typically connected to the surrounding tissue, the abdominal wall in the case of ventral hernias, with sutures. Each suture is generally a biologically compatible thread or fiber having generally opposed first and second ends. The suture is typically inserted by the health professional into the surrounding tissue from a second tissue surface, facing towards the health professional and generally opposite the first tissue surface, through the tissue and the first tissue surface and then through the patch. The suture is then drawn across a portion of a second sheet surface, generally opposite the first sheet surface, and then back through the sheet, the tissue, and the second tissue surface. Thus, there is an intermediate portion, intermediate the ends, extending across a portion of the second sheet surface. The suture, and more specifically the ends thereof, may then be pulled towards the health professional to ensure that the first sheet surface is held locally adjacently abutting the first tissue surface with the ends fastened together. This operation is generally repeated for each suture until the sheet is connected around the entirety of its perimeter to the surrounding tissue with the first sheet surface adjacently abutting the first tissue surface and a portion of the sheet completely covering the hernia. This technique is typically referred to as an underlay repair for a hernia, the advantages of which are well known to one skilled in the medical arts.

Unfortunately, as described above, the use of conventional patches for the underlay hernia repair technique described above obliges the health professional to insert the sutures through the tissue and the sheet of the patch, often with a needle, and then to loop the suture back through the sheet and tissue. As the sheet is placed on the first tissue surface facing away from the health professional, when the suture and needle are inserted through the sheet and tissue, they are often inserted towards subjacent internal organs, which creates a danger that the needle will pierce, and potentially damage, the subjacent internal organs. This may lead to surgical and post-surgical complications, such as, among others, tearing, bleeding (internal hemorrhage) of the internal organs such as intestine or the like and infection thereof (peritonitis, abscess). For example, in the case underlay repair of ventral hernias, the suture and needle are inserted towards the intestine, which poses a risk of damage thereto. Additionally, as the safe passage of the suture through the surrounding tissue and sheet requires careful manipulation of the needle to avoid other portions of non-damaged tissue, the use of conventional patches for the underlay procedure is also time consuming and complex.

Conventional installation of patches often leads to non-uniform and unequal attachment of the patch to the abdominal wall all around the hernia, which subsequently leads recurrent patch repair on a same patient.

Accordingly, there is a need for an improved prosthetic replacement patch and method of use thereof that obviate the aforementioned difficulties.

### SUMMARY OF THE INVENTION

It is therefore a general object of the present invention to provide an improved prosthetic replacement patch for repairing hernias in biological tissue of the abdominal wall or the like of a patient and a method therefor.

An advantage of the present invention is that repair of the hernia is simplified and accelerated by using the patch provided by the present invention.

Another advantage of the present invention is that the risk of piercing or damaging other tissue and subjacent internal organs during connection of the patch provided by the present invention to the tissue surrounding the hernia is reduced.

A further advantage of the patch provided by the present invention is that the risk of infection, either to the tissue surrounding the hernia or to other subjacent internal tissue, is reduced by use thereof to repair the hernia.

Still another advantage of the present invention is that the uniform and equal installation and attachment of the patch to the abdominal wall is increased while the risk of recurrence of the hernia is reduced.

Another advantage of the present invention is that the patch thereby allows for better placement of the patch compared to conventional placement of the patch.

According to a first aspect of the present invention, there is provided a prosthetic repair patch comprising:
a sheet comprising biologically compatible material, the sheet having first an second sheet surfaces and being sized and shaped for completely under covering an aperture in a biological tissue in a body of a patient with the first sheet surface adjacently abutting a first tissue surface of the tissue, the first tissue surface generally facing away from a person installing the patch and being generally opposed to a second tissue surface of the tissue; and
a plurality of retaining structures connected to the sheet in a spaced apart configuration from one another and extending from the first sheet surface, each retaining structure being configured for extension of a suture therethrough and for retaining the suture between respective generally opposed longitudinal first and second suture ends thereof, the suture being extendable through the tissue, from the second tissue surface through the first tissue surface, through the retaining structure, and back through the tissue from the first tissue surface through the second tissue with the first and second suture ends extending outwardly away from the second tissue surface for and being drawable outwardly away therefrom and attachable to one another to locally fasten the sheet to the tissue with the first sheet surface locally and adjacently abutting the first tissue surface.

In a second aspect of the present invention, there is provided a method for under covering an aperture in a biological tissue extending therearound in a body of a patient with a prosthetic repair patch comprising a sheet of biologically compatible material and a plurality of retaining structures connected thereto and extending from a first sheet surface of the sheet, each retaining structure being configured for extension of a suture therethrough and for retaining the suture between respective generally opposed longitudinal first and second suture ends thereof, the method comprising the steps of:
a) positioning the sheet proximal a first tissue surface of the tissue in the body with the first sheet surface facing the first tissue surface and the sheet extending under the aperture, the first tissue surface generally facing away from a person installing the patch and being generally opposed to a second tissue surface facing the person;
b) for each retaining structure, extending a respective said suture therefore from the second tissue surface through the first tissue surface, subsequently through the retaining structure, and back through the tissue from the first tissue surface through the second tissue with the first and second suture ends extending outwardly away from the second tissue surface;
c) for each respective said suture, pulling each said suture end until the first sheet surface locally and adjacently abuts the first tissue surface while under covering the aperture; and
d) for each respective said suture, attaching each said first and said second suture ends with another said suture end adjacent the second tissue surface to locally fasten the sheet to the tissue.

Other objects and advantages of the present invention will become apparent from a careful reading of the detailed description provided herein, with appropriate reference to the accompanying drawings.

### BRIEF DESCRIPTION OF THE DRAWINGS

Further aspects and advantages of the present invention will become better understood with reference to the description in association with the following Figures, in which similar references used in different Figures denote similar components, wherein:

FIG. 1 is a top perspective view of a prosthetic repair patch having an integrated retaining structure in accordance with an embodiment of the present invention.

FIG. 2 is side-sectional view of the patch shown in FIG. 1, taken along line 2-2 of FIG. 1 and showing a first configuration for a plate thereof; and

FIG. 2a is a view similar to FIG. 2, showing a second configuration for a plate thereof.

### DETAILED DESCRIPTION OF THE PREFERRED EMBODIMENTS

With reference to the annexed drawings the preferred embodiments of the present invention will be herein described for indicative purpose and by no means as of limitation.

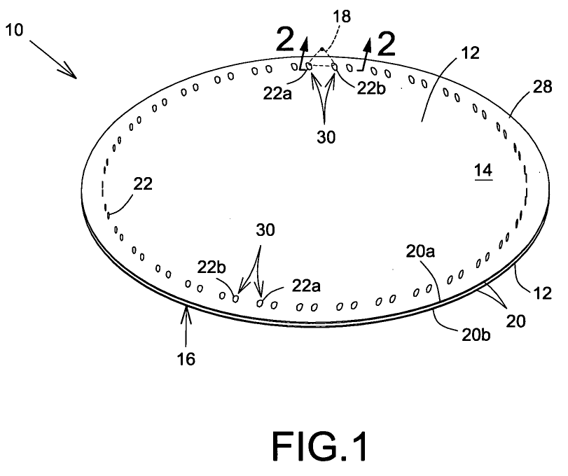

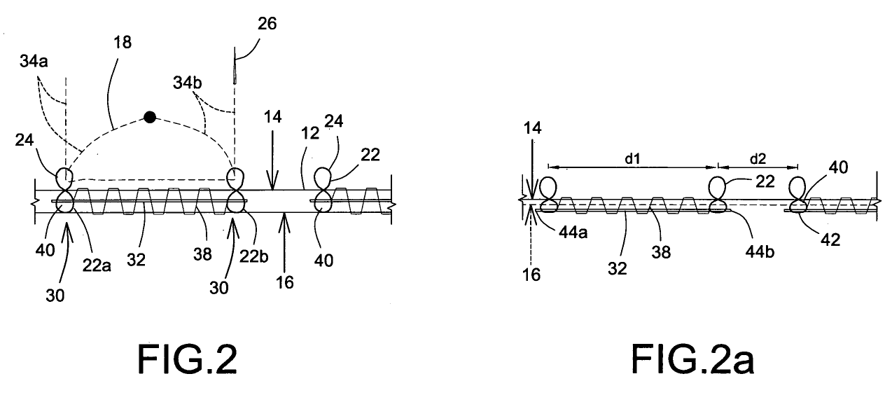

Reference is now made to FIGS. 1 and 2, which shows a prosthetic replacement patch, shown generally as 10, in accordance with an embodiment of the present invention for repairing an aperture, not shown, in surrounding biological tissue, not shown, of the abdominal wall of a patient. For the purposes of this description, it should be noted that the term aperture denotes any undesired aperture in biological tissue of a patient, including hernias, tears, punctures, and the like. However, the patch 10 described herein is ideally suited for repair of hernias, and ventral hernias in a particular, using an underlay repair surgical technique. It should also be noted that the term repair, with regard to apertures in the tissue, generally denotes, for the purposes of this description, the complete under covering of an aperture with the patch 10 and the connecting of the patch 10 to surrounding tissue surrounding the aperture, such that the aperture is completely covered, i.e. closed. However, the patch 10 described herein is particularly suited for use in underlay hernia repair procedures, in which the patch 10 is placed underneath the surrounding abdominal tissue surrounding the aperture, i.e. facing a first tissue surface, not shown, facing away from the health professional placing the patch in the patient, with the patch 10 completely under covering the aperture and sutured to the surrounding tissue on a second tissue surface, not shown, generally opposite the first tissue surface.

The patch 10 has a sheet 12, possibly consisting of two or more layers 20, and which has a first sheet surface 14 and a second sheet surface 16 comprised of biologically compatible material, suitable for placement within a patient. For example, for the embodiment shown, the patch 10 has a sheet 12 consisting of a first layer 20a and a second layer 20b, with the first sheet surface 14 being situated on the first layer 20a and the second sheet surface being situated on the second layer 20b, with the first and second sheet surfaces 14, 16 being generally opposite one another and facing away from one another. The biologically compatible materials for the sheet 12, and specifically the sheet surfaces 14, 16, typically consist of, for example, polyester, polyglycolic acid, polypropylene, polytetrafluoroethylene, and a combination of polytetrafluoroethylene and polypropylene. However, any biologically compatible material typically suitable for long term or permanent placement within a patient or which is eventually resorptive (absorbable) may be deployed, provided the material is suitable for under covering the aperture in the surrounding biological abdominal tissue. The sheet 12 is sized and shaped for completely covering the aperture in the surrounding biological tissue with the first sheet surface 14 adjacently and locally abutting the first tissue surface for closing off, i.e. covering, and repairing the aperture.

The patch 10 also has a plurality of integrated suture retaining, i.e. anchoring, structures 22, connected to the sheet 12 in a spaced apart configuration from each other, preferably around, or in proximity to, the entire perimeter 28 of the sheet 12. Each retaining structure 22 extends outwardly away from the first sheet surface 14 and is configured for passage of a suture 18 therethrough and retaining the suture 18 therein. Each suture 18 has generally opposed first and second longitudinal suture ends end 34a, 34b. The sutures 18 are used to connect the sheet 12 to the tissue, by passage of each suture through a retaining structure 22, to at least partially secure the sheet 12 thereto with the first sheet surface 14 adjacently abutting the first tissue surface for under covering the aperture. The sutures 18 are also made from biologically compatible materials, such as those mentioned for the sheet 12. The retaining structures are also made from biologically compatible materials, such as those mentioned for the sheet 12.

More specifically, each suture 18, typically a suture end 34a thereof, is passed, i.e. extended, through the tissue, from the second tissue surface to the first tissue surface, then through at least one retaining structure 22, and then back through the tissue from the first tissue surface to the second tissue surface such that both suture ends 34 extend outwardly away from the second tissue surface with the suture 18 extending through the tissue and at least one retaining structure 22. Once the suture 18 is extended through the tissue and structure 22 with the suture ends 34 extending away from the second tissue surface, the suture ends 34 may be drawn, i.e. pulled, away from the second tissue surface for locally and adjacently abutting the first sheet surface 14 to the first tissue surface. Each suture end 34 may then be attached to another suture end 34, typically of the same suture 18 but also possibly to another suture extended through another retaining structure as described above, adjacent the second tissue surface. Thus, the sutures 18, retained in the retaining structures 22, locally fasten the sheet 12 to the tissue with the first sheet surface 14 adjacently abutting the first tissue surface 24 for completely under covering, and thereby repairing, the aperture. Thus, the retaining structure 22 is configured for extension of a suture 18 therethrough and for retaining the suture 18, between the respective suture ends 34, connected to the sheet 12 when the suture 18 is extended through the tissue, from the second tissue surface through the first tissue surface, through the retaining structure, and back through the tissue from the first tissue surface through the second tissue surface with the first and second suture ends 34 extending outwardly away from the second tissue surface for attachment to one another to locally fasten the sheet 12 to the tissue with the first sheet surface 14 locally and adjacently abutting the first tissue surface. The extension of the suture 18 through the tissue and retaining structure 22, is typically accomplished using a suturing instrument 26, such as a surgical needle or the like, connected to a suture end 34.

Having described the patch 10 and its deployment generally, additional details on the embodiments shown are now provided, with reference to FIGS. 1, 2, and 2a. Each retaining structure 22 preferably includes a first, preferably closed and annularly shaped, portion 24 configured, i.e. sized and shaped, for passage of the suture 18 therethrough, as well as for passage therethrough of the suturing instrument 26 connected to one of the suture ends 34. However, provided the suture 18 and suturing instrument 26 may be passed therethrough, the first portion 24 could also leave an opening with respect to the first sheet surface 14 and need not necessarily be annular in shape. For example, the first portion 24 could also be shaped as a hook or the like connected to the sheet 12 and extending outwardly away from the first sheet surface 14.

As mentioned previously, the retaining structures 22 are, preferably, spaced apart relative one another around, or in proximity to, the perimeter 28 of the sheet 12. Preferably, but not compulsorily, the retaining structures 22 are further arranged into pairs, shown generally as 30, extending proximally around the perimeter 28, also in a spaced apart configuration relative one another.

Each pair 30 consists of a first retaining structure 22a and a second retaining structure 22b, spaced apart from one another at a distance d1 varying between about 0 mm and about 15 mm, the latter being the preferred distance for d1. Each pair 30 is also spaced apart from each other pair by a distance of d2 varying between about 0 mm and 5 mm, the latter being the preferred distance for d2. Further, when the retaining structures 28 are organized into pairs 30, a respective common suture 18 for each pair 30 is preferably passed through both retaining structures 22a, 22b of the pair 30. More specifically, the common suture 18 is extended, and more specifically a suture end 34 thereof using the suturing instrument 26, through the tissue from the second tissue surface through the first tissue surface, subsequently through the through the retaining structures 18, and notably the first, preferably annular, portions 24 thereof, and then back through the tissue from the first tissue surface through the second tissue surface. The suture ends 34 of the suture 18 are then pulled away from the second tissue surface to locally and adjacently abut the portion of the first sheet surface 14 on which the suture 18 extends in the retaining structures 22 against the first tissue surface. The suture ends 18 are then attached to one another adjacent the second tissue surface to partially locally connect the sheet 12 to the tissue with the portion of the first sheet surface 14 on which the suture 18 extends in the retaining structures 22 locally and adjacently abutting the first tissue surface. This procedure is repeated for all pairs 30 on the patch to locally connect the sheet 12 to the tissue with the first sheet surface 14 locally and adjacently abutting the first tissue surface.

To facilitate even and uniform placement and fixation of the patch 10, and particularly the first sheet surface 14 of the sheet 12 in local and adjacent abutment with the tissue around the aperture, when the retaining structures 22 are arranged in pairs 30, each pair 30, preferably, has a respective plate 32 extending longitudinally between the respective first and second retaining structures 22a, 22b and generally parallel to the sheet 12. The plate 32 is preferably connected to the retaining structures 22 and, thus, has a length similar to d1, i.e. between about 0 mm and about 15 mm. Further, the plate 32 preferably has a width of d3, varying between about 0 mm and about 2.5 mm, the latter being preferred for d3. The thickness of the plate 32 is, preferably between about 0 mm and about 1 mm, the latter being the preferred thickness. The plate 32 is made of rigid material, such as plastic, metal or the like. The plate 32 may be situated, extending longitudinally between the retaining structures 22a, 22b of pair 30, between the first and second sheet surfaces 14, 16 as shown in FIG. 2. Alternatively, the plate 32 may be situated, extending longitudinally between the retaining structures 22a, 22b of pair 30, on either of the first and second sheet surfaces 14, 16, preferably in proximity to or abutment therewith, for example as shown, with regard to the second sheet surface 16, in FIG. 2a.

The plate 32 is, preferably, connected to the sheet 12, as shown in FIGS. 2 and 2a, by a string or plate connecting suture 38, also of biocompatible material, sewn through the plate 32, and notably through plate apertures, not shown, therein, and at least one sheet surface 14, 16, during manufacture of the patch 10. Each plate connecting suture 38 is, preferably, connected to the first and second retaining structures 22a, 22b between which the plate 32 extends, thereby connecting the plate 32 thereto.

To ensure secure connection of the retaining structure 22 to the sheet 12, each retaining structure 22 also has a second, sheet connecting portion 40 extending from the first portion 24 into the sheet 12. It is, preferably, to this second portion 40 of each retaining structure 22a, 22b of each pair 30 that the plate 32, preferably at generally opposed first and second longitudinal plate ends 44a, 44b thereof, is connected, either directly or, preferably, by using the plate connecting suture 38 sewn to the plate 32 and connected to the second portion 40. As shown in FIGS. 2 and 2a, the second portion 40 is, preferably, annular in shape, like the first portion 24, with the plate 32 extending therethrough. Thus, the two portions 24, 40, in their preferred configuration, preferably form a figure-eight configuration, again as shown in FIGS. 2 and 2a, with the first half of the figure-eight, i.e. the first portion 24 extending outwardly away from the first surface 14 and the second half of the figure-eight, i.e. the second portion, extending into the sheet 12. The second portion 40, regardless of its shape, is connected to the first portion 24 and extends into the sheet 12, to which the second portion 40 is securely affixed, thus securely connecting the retaining structure 22 to the sheet 12. The second portion 40 may extend between the first and second sheet surfaces 14, 16 and be situated entirely therebetween, as shown in FIG. 2. Alternatively, and as shown in FIG. 2a, the second portion 40 may extend from within the sheet 12, i.e. between the two sheet surfaces 14, 16, out of the second sheet surface 16, i.e. a protruding segment 42 of the second portion 40 may extend outwardly through the second sheet surface 16, as shown in FIG. 2a. The configuration in FIG. 2a is, notably, preferred when the plate 32 extends upon the second sheet surface 16. It should be noted that, while the second portion 40 is preferably annular in shape, the second portion may have any other shape, provided that the shape allows the second portion 40 to securely connect the first portion 24 to the sheet 12.

While the distances for the spacing of the retaining structures 22 and the size of the plates 32 described herein are well adapted for use of the patch 10 to repair apertures such as ventral hernias, the spacing may be adapted, i.e. modified, in function of the size of the sheet 12 as well as the size of the aperture to be repaired. For example, larger apertures may require larger sheets and greater, or less, spacing between retaining structures 22, as well as larger or smaller sizes for the plates 32.

Having described the patch 10 generally, the deployment thereof for use in an underlay repair procedure for an aperture in tissue, such as a ventral hernia, is now described. Initially, the patch 10 is positioned with the sheet 12, and preferably the first sheet surface 14, proximal the first tissue surface and extending under and toward the aperture. For each retaining structure 18 or, when the retaining structures 22 are arranged in pairs 30, each pair of retaining structures, a suture 18, and more specifically a suture end 34 of each suture 18, is then extended, i.e. drawn with the suturing instrument 26 attached to the suture end 34, through the tissue, from the first tissue surface therethrough and out of the second tissue surface. The suture 18, and more specifically the suture end 34 having the suturing instrument 26 attached thereto, is then extended through the retaining structure 22, or, when the retaining structures 22 are arranged in pairs, through the first and second retaining structures 22a, 22b. The suture 18 is then extended, i.e. drawn with the suturing instrument 26 attached to the suture end 34, back through the tissue, from the second tissue surface therethrough and out of the first tissue surface. This process is repeated for each retaining structure 22 or pair 30 of retaining structures 22. Each suture end 34a, 34b of each suture is then pulled until the first sheet surface 14 locally and adjacently abuts the first tissue surface while covering the aperture. Suture ends 34a, 34b (preferably of a same suture 18) are then attached to one another adjacent the second tissue surface to locally fasten the sheet 12 to the tissue with the sheet 12, and notably the first sheet surface 14, under covering the aperture.

Advantageously, since the retaining structures 22, and particularly the respective first portions 24 thereof, by which the sutures 18 are connected to the sheet and retained, are already connected to the sheet 12 and extend outwardly away from the first sheet surface 14. Accordingly, there is no need, unlike with conventional patches, to use the suturing instrument 26 or other surgical tools to thread the suture 18 from the first sheet surface 14 through the sheet 12, and possibly out through the second sheet surface 16, and then back through the sheet 12 out of the first sheet surface 14 to connect the suture to the sheet 12. Accordingly, the surgical procedure of repairing the aperture with the patch 10 of the present invention is facilitated and the amount of time required to perform the procedure, compared to conventional patches, is reduced. Further, the risk of damaging other tissue or internal organs in proximity to the surrounding tissue by inserting the suturing instrument 26 or other instrument through the patch 10, as required with conventional patches, is eliminated. The elimination of this risk also reduces the risk of infection and of complications. In addition, as the retaining structures 22 are already connected to the patch 10 in a spaced apart relationship around the perimeter 28, the risk of irregular stitching, non-uniform placement or attachment of the sutures 18 to the patch 10 and tissue, which may be encountered with conventional patches, is reduced and proper placement of the patch 10 relative the tissue and aperture is facilitated.

Although the present patch has been described with a certain degree of particularity, it is to be understood that the disclosure has been made by way of example only and that the present invention is not limited to the features of the embodiments described and illustrated herein, but includes all variations and modifications within the scope and spirit of the invention as hereinafter claimed.

I claim:

1. A prosthetic repair patch comprising:
a sheet comprising biologically compatible material, said sheet having first an second sheet surfaces and being sized and shaped for completely under covering an aperture in a biological tissue in a body of a patient with said first sheet surface adjacently abutting a first tissue surface of the tissue, the first tissue surface generally facing away from a person installing said patch and being generally opposed to a second tissue surface of the tissue; and
a plurality of retaining structures connected to said sheet in a spaced apart configuration from one another and extending from said first sheet surface, each retaining structure being configured for extension of a suture therethrough and for retaining the suture between respective generally opposed longitudinal first and second suture ends thereof, the suture being extendable through the tissue, from the second tissue surface through the first tissue surface, through said retaining structure, and back through the tissue from the first tissue surface through the second tissue with the first and second suture ends extending outwardly away from the second tissue surface for and being drawable outwardly away therefrom and attachable to one another to locally fasten said sheet to the tissue with said first sheet surface locally and adjacently abutting the first tissue surface.

2. The prosthetic repair patch of claim 1, wherein said retaining structures are connected to sa id sheet in proximity to a perimeter thereof.

3. The prosthetic repair patch if claim 3, wherein said retaining structures are spaced apart from one another extending around said perimeter.

4. The prosthetic repair patch of claim 1, wherein said retaining structure is comprised of one of polyester, polyglycolic acid, polypropylene, polytetrafluoroethylene, and a combination of polytetrafluoroethylene and polypropylene.

5. The prosthetic repair patch of claim 1, wherein each retaining structure comprises an annular first portion connected to said sheet and extending from said first sheet surface, said first portion being annularly shaped and configured for passing therethrough of a suturing instrument having one of the first and second suture ends connected thereto for extending the suture through the tissue and said first portion.

6. The prosthetic repair patch of claim 1, wherein said retaining structures are arranged in pairs, the suture being extendable, between the first and second suture ends, through each retaining structure of said pair.

7. The prosthetic repair patch of claim 6, further comprising, for each said pair, a respective plate connected to said sheet and to each retaining structure of said pair and extending longitudinally between each retaining structure of said pair.

8. The prosthetic repair patch of claim 7, wherein each pair is spaced apart relative each other at a first distance varying between about 0 mm and about 5 mm.

9. The prosthetic repair patch of claim 8, wherein each retaining structure of each pair is spaced apart relative one another at a second distance varying between about 0 mm and about 15 mm.

10. The prosthetic repair patch of claim 9, wherein said plate is between about 1 mm and 2.5 mm in thickness.

11. The prosthetic repair patch of claim 9, wherein said plate is less than about 2.5 mm in width.

12. The prosthetic repair patch of claim 7, wherein said plate is situated between said first and said second sheet surfaces.

13. The prosthetic repair patch of claim 7, wherein said plate is situated on one of said first sheet surface and said second sheet surface.

14. The prosthetic repair patch of claim 7, wherein said plate is sewn to said sheet.

15. The prosthetic repair patch of claim 7, wherein each retaining structure comprises a second portion fixedly connected to said sheet and extending therein through at least one of said first sheet surface and said second sheet surface, said respective plate being connected proximal a respective first plate end thereof to said second portion of one said retaining structure of said pair and connected proximal a respective second plate end thereof, generally longitudinally opposed to said first plate end, to said second portion of another said retaining structure of said pair.

16. The prosthetic repair patch of claim 15, wherein said second portion is annular in shape.

17. The prosthetic repair patch of claim 7, wherein said plate is comprised of a metal.

18. A method for under covering an aperture in a biological tissue extending therearound in a body of a patient with a prosthetic repair patch comprising a sheet of biologically compatible material and a plurality of retaining structures connected thereto and extending from a first sheet surface of said sheet, each retaining structure being configured for extension of a suture therethrough and for retaining the suture between respective generally opposed longitudinal first and second suture ends thereof, said method comprising the steps of:
a) positioning said sheet proximal a first tissue surface of the tissue in the body with said first sheet surface facing the first tissue surface and said sheet extending under the aperture, the first tissue surface generally facing away from a person installing said patch and being generally opposed to a second tissue surface facing the person;
b) for each retaining structure, extending a respective said suture therefor from the second tissue surface through the first tissue surface, subsequently through said retaining structure, and back through the tissue from the first tissue surface through the second tissue with the first and second suture ends extending outwardly away from the second tissue surface;
c) for each respective said suture, pulling each said suture end until said first sheet surface locally and adjacently abuts the first tissue surface while under covering the aperture; and
d) for each respective said suture, attaching each said first and said second suture ends with another said suture end adjacent the second tissue surface to locally fasten said sheet to the tissue.

19. The method of claim 18, wherein the retaining structures are organized into pairs of respective first and second retaining structures spaced apart relative one another and having a same respective said suture therefore, the suture being extendable through both said respective first and second retaining structures during the step of extending a respective said suture.
